(19) United States
(12) Patent Application Publication
(10) Pub. No.: US 2019/0008629 A1
(43) Pub. Date: Jan. 10, 2019

(54) SCAFFOLD DELIVERY

(71) Applicant: Abbott Cardiovascular Systems Inc., Santa Clara, CA (US)

(72) Inventors: Erik D. Eli, Redwood City, CA (US); Senthil Eswaran, Sunnyvale, CA (US); Denis Tauz, Sunnyvale, CA (US); Michael L. Green, Pleasanton, CA (US)

(21) Appl. No.: 15/643,872

(22) Filed: Jul. 7, 2017

(51) Int. Cl. A61F 2/04; A61F 2/24; A61F 2/958

(52) U.S. Cl. CPC A61F 2/04; A61F 2/2433; A61F 2/86; A61F 2/2418; A61F 2/958

(57) ABSTRACT

A catheter assembly is configured to segmentally expand a scaffold. The catheter assembly includes a balloon that is inflated to expand a first scaffold region, is deflated and retracted into the expanded first scaffold region, and then inflated to expand a second scaffold region.

### FIELD

This disclosure relates generally to medical devices and more particularly to a catheter for delivering an endoprosthesis.

### BACKGROUND

Radially expandable endoprostheses are artificial devices adapted to be implanted or deployed in an anatomical lumen. An “anatomical lumen” refers to a cavity, duct, of a tubular organ such as a blood vessel, urinary tract, and bile duct. Stents are examples of endoprostheses that are generally cylindrical in shape and function to hold open and sometimes expand a segment of an anatomical lumen. Stents are often used in the treatment of atherosclerotic stenosis in blood vessels. “Stenosis” refers to a narrowing or constriction of the diameter of an anatomical lumen or orifice. In such treatments, stents reinforce the walls of the blood vessel and may prevent restenosis (a recurrence of the stenosis) following an angioplasty procedure.

The treatment of a diseased site or lesion with a stent involves both delivery and deployment of the stent. “Delivery” refers to introducing and transporting the stent through an anatomical lumen to the diseased site or lesion located at a target region of the anatomical lumen. “Deployment” corresponds to expansion of the stent within the lumen at the target region. Delivery and deployment of a stent are accomplished by positioning the stent about one end of a catheter, inserting the end of the catheter through the skin into an anatomical lumen, advancing the catheter in the anatomical lumen to the target region, expanding the stent at the target region, and then removing the catheter from the target region.

A self-expanding stent is capable of expanding from a compressed or collapsed state to a radially expanded state. A delivery device, such as a catheter assembly, which retains the stent in its compressed state is used to deliver the stent to the target region. After the stent is positioned at the target region, the delivery device is actuated to release the stent which allows the stent to self-expand within the target region. The delivery device is then detached from the stent and removed from the target region while the stent remains at the target region.

The stent should be able to satisfy a number of basic, functional requirements. The stent should be capable of withstanding the structural loads, for example, radial compressive forces, imposed on the stent as it supports the walls of a vessel after deployment. Therefore, a stent should possess adequate radial strength. After deployment, the stent should adequately maintain its size and shape throughout its service life despite the various forces that may come to bear on it. In particular, the stent should adequately maintain a vessel at a prescribed diameter for a desired treatment time despite these forces. The treatment time may correspond to the time required for the vessel walls to remodel, after which the stent is no longer necessary.

### SUMMARY

Briefly and in general terms, the invention is directed to catheter system, a method of implanting a scaffold, and a method of deploying a scaffold.

In aspects of the invention, a catheter system comprises a hollow tube forming a sheath, a balloon configured to slide out of and into the sheath, and a braid of polymer filaments forming a scaffold. The scaffold includes a first scaffold region and a second scaffold region located rearward of the first scaffold region. The scaffold has a covered state when fully inside the sheath, a partially covered state when the first scaffold region is outside the sheath and the second scaffold region is inside the sheath, and a non-covered state when fully outside the sheath. When in the covered state, the first scaffold region and the second scaffold region are disposed within the sheath and each have starting diameters. When in the partially covered state, the first scaffold region is disposed outside of the sheath and connected to the second scaffold region, the second scaffold region is disposed within the sheath, and the balloon is configured to inflate while outside of the sheath such that inflation expands the first scaffold region to a diameter greater than the starting diameter of the first scaffold region.

In aspects of the invention, a method of implanting comprises inserting the catheter system into an anatomical lumen, followed by retracting the sheath to expose the first region of the scaffold, followed by inflating the balloon to expand the first region while the second region of the scaffold is disposed within the sheath, followed by retracting the sheath to expose the second region, followed by inflating the balloon to expand the second region, followed by withdrawing the catheter assembly from the anatomical lumen while the first and second regions remain attached to each other and remain in the anatomical lumen.

In aspects of the invention, a method of deploying comprises retracting a sheath by a first retraction distance in a rearward direction to expose a first region of a scaffold, followed by inflating a balloon to expand the first region while a second region of the scaffold is disposed in the sheath, followed by retracting the sheath by a second retraction distance in the rearward direction to expose the second region, followed by inflating the balloon to expand the second region while the second region remains connected to the first region.

The features and advantages of the invention will be more readily understood from the following detailed description which should be read in conjunction with the accompanying drawings.

### BRIEF DESCRIPTION OF THE DRAWINGS

FIG. 1A is a side view of an example scaffold.

FIG. 1B is a side view of the scaffold of FIG. 1A after having been loaded into a sheath illustrated in cross-section.

FIG. 1C is a side view of the scaffold of FIG. 1B after having been released from the sheath.

FIG. 1D is a side view of the scaffold of FIG. 1C with a balloon placed within the scaffold.

FIG. 2 is a schematic plan view of an example catheter system, partially showing a rear segment of a catheter assembly and partially showing a front segment of the catheter assembly with a balloon and the scaffold of FIG. 1C. The rear and front segments of the catheter assembly are connected, as indicated by the broken line.

FIG. 3A is a cross-section view showing an example balloon of the catheter assembly before inflation of the balloon.

FIG. 3B is a cross-section view of the balloon of FIG. 3A after being inflated.

FIG. 3C is a cross-section view of the balloon of FIG. 3B after being deflated.

FIG. 4A is a cross-section view showing another example balloon of the catheter assembly before inflation of the balloon.

FIG. 4B is a cross-section view of the balloon of FIG. 4A after being inflated.

FIG. 4C is a cross-section view of the balloon of FIG. 4B after being deflated.

FIGS. 5A-5L are cross-section views of an anatomical lumen, showing the front segment of the catheter assembly of FIG. 2 being used for segmental expansion of the scaffold. FIGS. 5A-5L show an example sequence of events during segmental expansion.

FIG. 6 is a schematic plan view of another example catheter system, partially showing a rear segment of a catheter assembly and partially showing a front segment of the catheter assembly with a balloon and the scaffold of FIG. 1C. The rear and front segments of the catheter assembly are connected, as indicated by the broken line.

FIGS. 7A-7L are cross-section views of an anatomical lumen, showing the front segment of the catheter assembly of FIG. 6 being used for segmental expansion of the scaffold. FIGS. 7A-7L show an example sequence of events during segmental expansion.

FIG. 8 is a cross-section view of an example balloon wall used for segmental expansion of the scaffold.

FIGS. 9-11 are enlarged cross-section views of the balloon wall of FIG. 8, showing various surface features on the balloon wall.

FIGS. 12 and 13 are side views showing example pulling devices adjacent the balloon for exposing the scaffold.

### DETAILED DESCRIPTION

As used herein, the term “axial” and the like refer to a direction, orientation, or line that is parallel or substantially parallel to the central axis of a cylindrical or tubular construct and is sometimes used to refer to movement. The term “radial” and the like refer to a direction, orientation, or line that is perpendicular or substantially perpendicular to the central axis of a cylindrical or tubular construct and is sometimes used to refer expansion.

As used herein, the term “braid” encompasses various braid patterns and weave patterns. Braid patterns include without limitation full diamond, half diamond, and herringbone patterns described in US Patent Application Publication No. 2015/0081000, which is incorporated herein.

As used herein, the term “filament” refers to an elongate structure and encompasses a fiber, strand, and ribbon. A single fiber, strand, or ribbon may form a filament. Multiple fibers, stands or ribbons may be joined, such as by twisting or fusing, to form a filament. A filament may have a circular, rectangular or irregular cross-section.

The scaffold described is a stent. The scaffold may be formed of bioresorbable material. As used herein, the term “bioresorbable” refers to the property of a material or endoprosthesis to degrade, absorb, resorb, or erode away from an implant site or target region of an anatomical lumen. The bioresorbable scaffold is intended to remain in a patient's body for only a limited period of time. In many treatment applications, the presence of an endoprosthesis in a body may be necessary for a limited period of time until its intended function of, for example, maintaining vascular patency and/or drug delivery is accomplished. Moreover, it has been shown that bioresorbable scaffolds may allow for improved healing of the anatomical lumen as compared to metal devices, which may lead to a reduced incidence of late stage thrombosis.

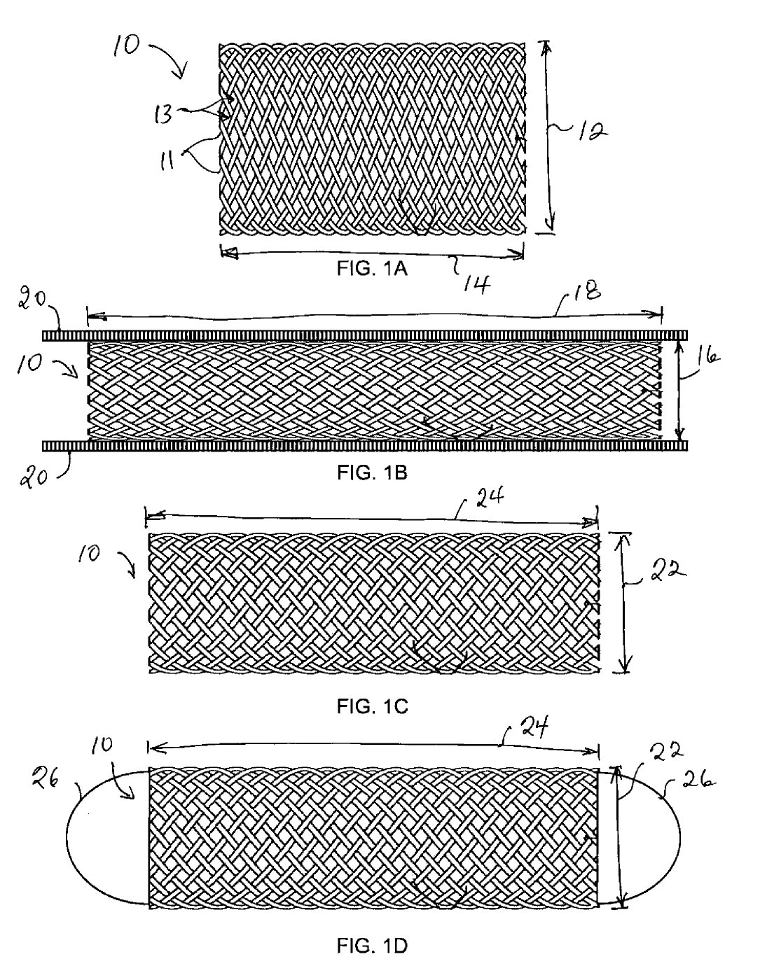

Referring now in more detail to the example drawings for purposes of illustrating aspects of the invention, wherein like reference numerals designate corresponding or like elements among the several views, there is shown in FIG. 1A example scaffold 10 that is flexible and designed to be compressed to a compressed diameter to allow it to be more easily delivered through an anatomical lumen using a catheter. Scaffold 10 is a hollow tube that includes a cylindrical wall formed by a braid of filaments 11 that cross over and cross under each other. There may be air gaps 13 between filaments 11.

Scaffold 10 has nominal diameter 12 and nominal axial length 14 before being mounted on a catheter. Nominal diameter 12 may be from 4 mm to 12 mm. Other diameters are possible. Nominal axial length 14 may be from 6 mm to 100 mm. Other lengths are possible. The diameter and length of scaffold 10 are defined by the cylindrical wall formed by the braid of filaments 11. The diameter of scaffold 10 corresponds to an outer diameter of the cylindrical wall formed by the braid of filaments 11. The cylindrical wall may have a cross-section that forms a circle, ellipse, oval, or other shape. As used herein, the term “diameter” is not limited to a circle and refers to the greatest width (outer surface to opposite outer surface) of the cylindrical wall.

In FIG. 1B, scaffold 10 has been radially compressed to compressed diameter 16. When radially compressed, scaffold 10 lengthens to elongated axial length 18 which is greater than nominal axial length 14. Scaffold 10 becomes longer due to the braided construction of scaffold 10. The amount of length elongation will depend on the amount of diameter compression. For example, extended axial length 18 may be over 200%, over 400%, or over 500% of nominal axial length 14. Scaffold 10 is constrained at compressed diameter 16 within a tube structure, referred to as sheath 20. Scaffold 10 is resilient so that when it is released from sheath 20, scaffold 10 may self-expand to nominal diameter 12 and return to the configuration of FIG. 1A.

It has been found that a scaffold made of a braid of certain polymer materials, such as materials containing PLLA or other bioabsorbable polymer, may experience the phenomenon of stress relaxation. Stress relaxation occurs, scaffold 10 may be unable to self-expand to nominal diameter 12 when released from sheath 20. Its ability to self-expand to nominal diameter 12 will depend on the amount of stress relaxation that has occurred.

As shown in FIG. 1C, scaffold 10 may self-expand to intermediate diameter 22 after being released from within sheath 20. Intermediate diameter 22 is greater than compressed diameter 16 and less than nominal diameter 12. Scaffold 10 does not return fully to the configuration of FIG. 1A unless a radially outward pressure is applied to the scaffold. The actual dimension of intermediate diameter 22 may depend on the amount of stress relaxation that has occurred. For example, intermediate diameter 22 may be less than 80% or less than 50% of nominal diameter 12 of FIG. 1A. Nominal diameter 12 can be a desired diameter. In some instances, intermediate diameter 22 could be insufficient for producing a desired therapeutic effect within a patient.

In FIG. 1D, an inflatable device, referred to as balloon 26, is inserted within scaffold 10. Balloon 26 may be inflated to an inflation diameter that is from 80% to 120% of nominal diameter 12. When balloon 26 is deflated and removed, scaffold 10 could be expected to maintain a deployed diameter that is 90% to 100% of the inflation diameter.

As mentioned above, a change in the diameter of scaffold 10 is accompanied by an opposite change in the axial length of scaffold 10. To expand from intermediate diameter 22 to nominal diameter 12, scaffold 10 will tend to shorten axially from intermediate axial length 24 to nominal axial length 14. Frictional engagement between balloon 26 and the inner surfaces of scaffold 10 may impede or prevent shortening of scaffold 10, which may make it difficult to expand scaffold 10 from intermediate diameter 22 to nominal diameter 12.

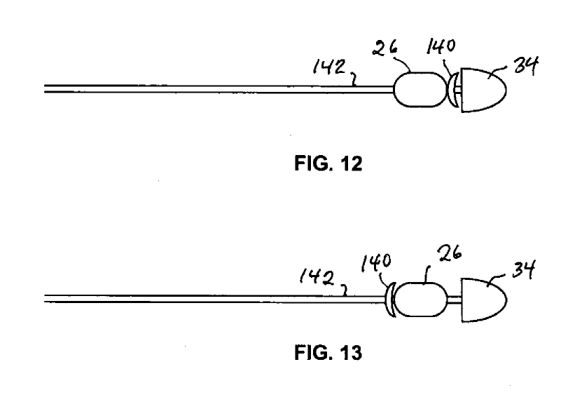

In use, scaffold 10 may be deployed and then expanded. Deployment involves exposing scaffold 10 out from sheath 20. This may be accomplished by pulling scaffold 10 out of sheath 20. For example, balloon 26 may frictionally engage a forward region of scaffold 10 to pull scaffold 10 out of sheath 20. Additionally or alternatively, a pulling device (for example, device 140 in FIGS. 12 and 13) adjacent to the balloon (in front or behind the balloon) may frictionally engage a forward region of scaffold 10 to pull scaffold 10 out of sheath 20. Additionally or alternatively, a driver (for example, driver 36 in FIG. 36 may push a rear region of the balloon so that scaffold 10 is exposed out from sheath 20.

Another approach to address stress relaxation is to segmentally expand scaffold 10 once deployed from the sheath. Unlike FIG. 1D, the entire axial length of scaffold 10 is not expanded simultaneously during segmental expansion. During segmental expansion, regions of scaffold 10 are exposed and expanded separately and sequentially, as will be described below. Segmental expansion may help ensure that scaffold 10 deploys at the desired target region.

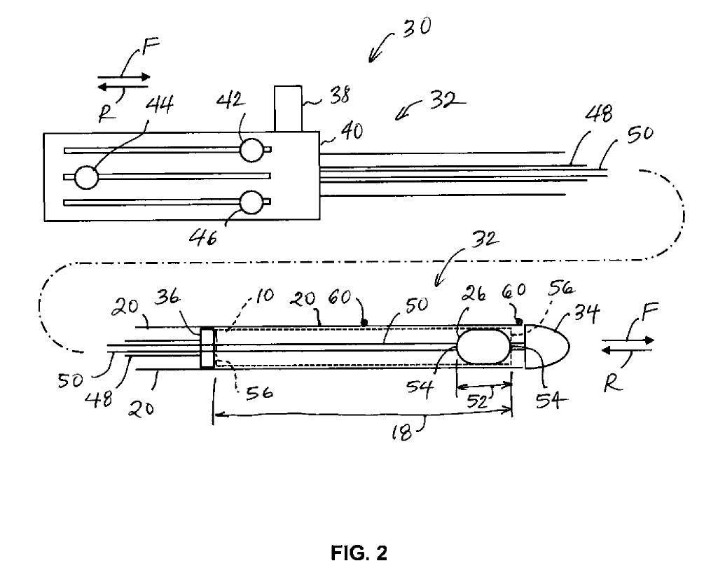

FIG. 2 shows example catheter system 30 comprising catheter assembly 32 and scaffold 10. Catheter assembly 32 is configured for segmental expansion of scaffold 10. Catheter assembly 32 includes catheter tip 34, balloon 26, sheath 20, and driver 36 at the front segment of the catheter assembly. Scaffold 10 is in a covered state in that all regions of scaffold 10 are within sheath 20. Catheter assembly 32 includes fluid port 38 to which a syringe may be connected to inflate and deflate balloon 26.

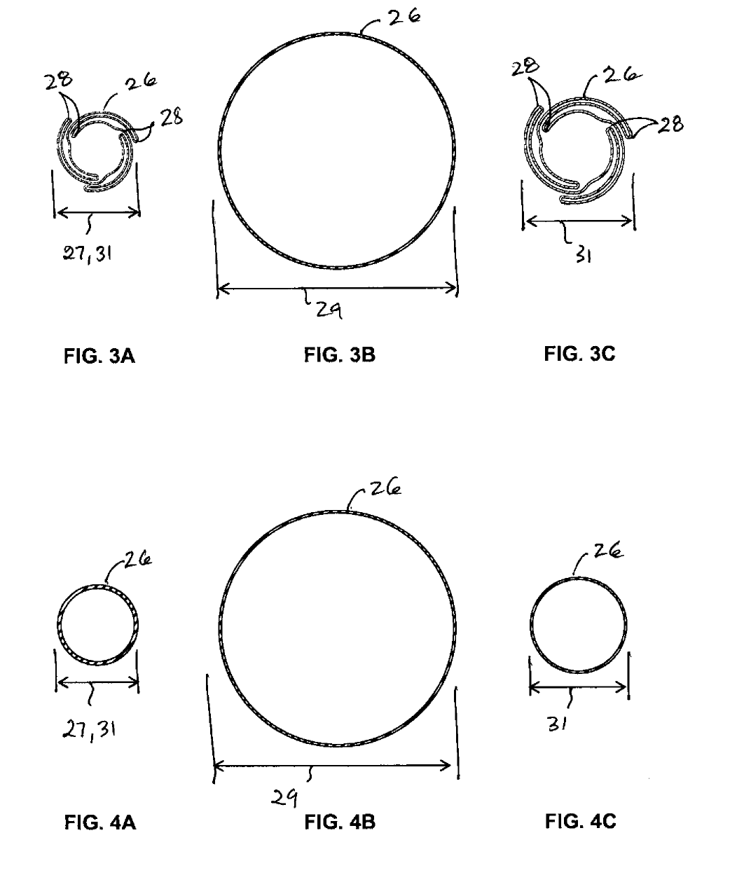

As shown in FIG. 3A, balloon 26 has starting diameter 27 and includes pleats 28 to facilitate inflation and subsequent deflation into a compact shape. Starting diameter 27 may be the same as the diameter of balloon 26 when balloon 26 is within sheath 20. As shown in FIG. 3B, pleats 28 unfold when balloon 26 is fully inflated to inflated diameter 29 when balloon 28 is outside of sheath 20. Inflated diameter 29 is the diameter needed to make scaffold 10 maintain a desired deployed diameter within the patient after balloon 26 is withdrawn from scaffold 10. For example, balloon inflated diameter 29 may be from 80% to 120% of scaffold nominal diameter 12. When balloon 26 is deflated, pleats 28 fold so that balloon 26 achieves deflated diameter 31. When deflated, balloon 26 goes to the configuration of FIG. 3A or the configuration of FIG. 3C. In FIG. 3A, balloon 26 has deflated diameter 31 which is equal to starting diameter 27. In FIG. 3C, balloon 26 has deflated diameter 31 which is greater (for example, at least 10% greater) than starting diameter 27, which may be due to plastic deformation of the balloon material during inflation and/or an inability to fold in precisely the same manner as in FIG. 3A.

In some aspects, balloon 26 does not have pleats that fold. In FIGS. 4A-4C, balloon 26 is made of balloon material having sufficient elasticity that allows balloon 26 to expand from starting diameter 27 to inflated diameter 29. Balloon materials which may have sufficient elasticity include C-Flex (R) thermoplastic elastomer from Saint-Gobain Performance Plastics of Clearwater, Fla. Another suitable material includes silicone rubber. Other materials may be used. When balloon 26 is deflated after inflation, balloon 26 achieves deflated diameter 31 without folding. Balloon 26 does not fold and it returns to the configuration of FIG. 4A or the configuration of FIG. 4C. In FIG. 4C, balloon 26 has deflated diameter 31 which is equal to starting diameter 27. In FIG. 4C, balloon 26 has deflated diameter 31 which is greater (for example, at least 10% greater) than starting diameter 27, which may be due to plastic deformation during balloon inflation.

Referring again to FIG. 2, catheter assembly 32 includes control device 40 at the rear segment of the catheter assembly. Control device 40 includes knobs 42, 44, 46 that the user may slide to control movement of various parts at the front segment of the catheter assembly.

Sheath knob 42 is operatively coupled to sheath 20. Sliding sheath knob 42 in rearward direction R and forward direction F causes sheath 20 to move axially in rearward direction R and forward direction F, respectively, relative to scaffold 10. The forward and rearward movements of sheath 20 may be relative to balloon 26 and/or driver 36. The forward and rearward movements of sheath 20 may be performed independently of movements of balloon 26 and/or driver 36.

Driver knob 44 is operatively coupled to driver 36. Sliding driver knob 44 in rearward direction R and forward direction F causes driver 36 to move axially in rearward direction R and forward direction F, respectively. Driver connector 48 connects driver knob 44 to driver 36. Driver connector 48 may include a driver wire or driver tube secured to driver 36. The forward and rearward movements of driver 36 may be relative to balloon 26 and/or sheath 20. The forward and rearward movements of driver 36 may be performed independently of movements of balloon 26 and/or sheath 20.

Balloon knob 46 is operatively coupled to balloon 26. Sliding balloon knob 46 in rearward direction R and forward direction F causes balloon 26 to move axially in rearward direction R and forward direction F, respectively. Balloon connector 50 connects balloon knob 46 to balloon 26. The forward and rearward movements of balloon 26 may be relative to sheath 20 and/or driver 36. The forward and rearward movements of balloon 26 may be performed independently of movements of sheath 20 and/or driver 36.

Balloon connector 50 may include a wire or tube secured to balloon 26. For example, balloon connector 50 may include an inflation tube that conveys inflation fluid from fluid port 38 to balloon 26. The inflation tube or other balloon connector may be contained within a driver tube of driver connector 48.

Control device 40 of catheter assembly 32 may be designed in other ways. For example, control device 40 may include gears that convert rotation to linear movement. The gears are connected to any of knobs 42, 44, 46 such that the user may rotate the knob(s) to control movement of various parts at the front segment of the catheter assembly.

As shown in FIG. 2, balloon 26 has axial length 52 that is less than compressed axial length 18 of scaffold 10. For example, balloon axial length 52 may be less than 50%, less than 20%, or less than 10% of compressed axial length 18. Balloon axial length 52 is defined by the axial distance between opposite ends (forward and rear ends) 54 of balloon 26 which are capable of expanding. Scaffold axial length 18 is defined by the axial distance between opposite ends (forward and rear ends) 56 of scaffold 10. The relatively short length of balloon 26 facilitates segmental expansion of scaffold 10 from intermediate diameter 22 to nominal diameter 12. Also, the relatively short length of balloon 26 may reduce difficulties in scaffold expansion arising from friction with the balloon, and/or increase reliability in scaffold deployment at the target region.

In use, a guidewire may first be inserted into an anatomical lumen. The forward end of the guidewire is maneuvered beyond the region of the anatomical lumen where it is desired to implant scaffold 10. That region is referred to as the target region. The rear end of the guidewire remains outside the patient and is fed into an opening in catheter tip 34. Next, the forward end of catheter assembly 32 is pushed into the anatomical lumen. The guidewire serves to guide the forward end of catheter assembly 32 to the target region.

Fluoroscopic techniques may be used by the clinician to visualize movement of the forward end of catheter assembly 32. Sheath 20 may include radiopaque markers 60 that are visible under fluoroscopy. Radiopaque markers 60 contain material (such as gold, tungsten, or a platinum/iridium alloy) having a radiopacity that is greater than that of surrounding parts of sheath 20. The difference in radiopacity creates a visual marker that helps a clinician determine the position of the forward end of catheter assembly 32 relative to the target region. The forward end of catheter assembly 32 is pushed forward until radiopaque markers 60 reach the target region, which is where scaffold 10 is to be completely released from sheath 20 and expanded by balloon 26. Although radiopaque markers 60 are illustrated schematically as protruding from the outer surface of sheath 20, radiopaque markers 60 may be flush with the outer surface of sheath 20 and/or may be imbedded so as not to protrude from the outer surface of sheath 20.

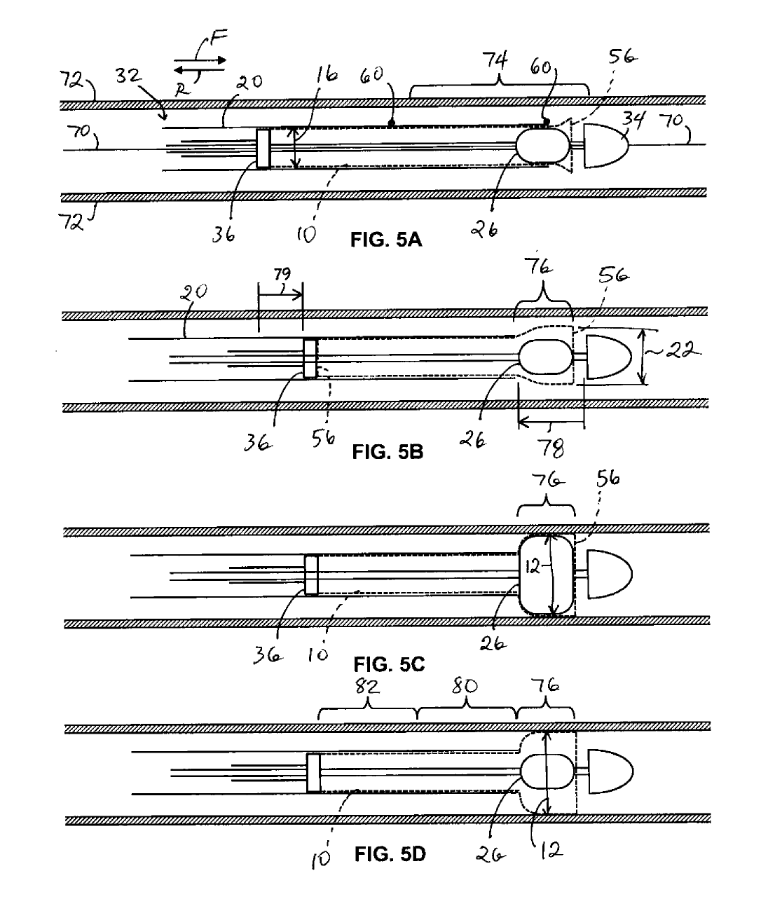

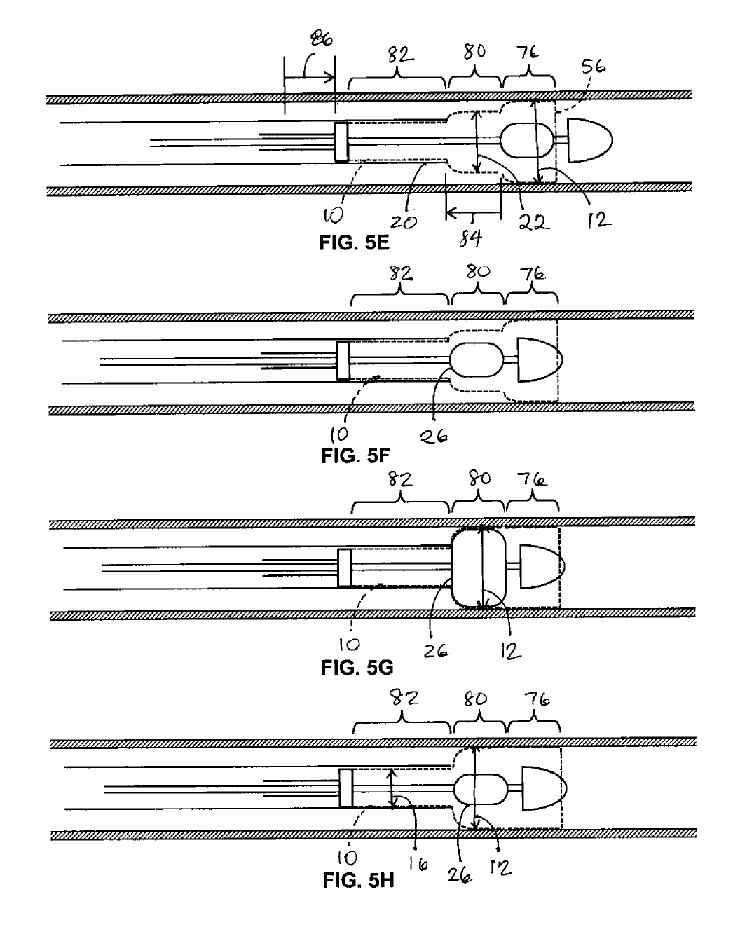

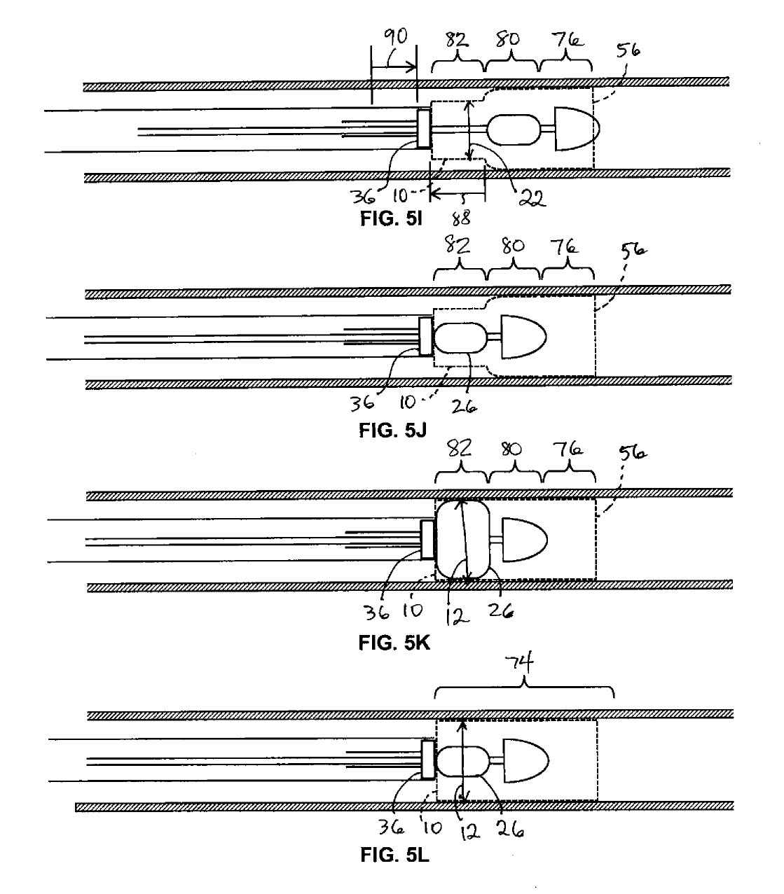

FIGS. 5A-5L illustrate an example process for segmental expansion of scaffold 10 using catheter assembly 32 of FIG. 2. In FIG. 5A, guidewire 70 was inserted in anatomical lumen 72 and has guided the forward end of catheter assembly 32 to target region 74. Although not illustrated, target region 74 may have a stenosis, lesion, or other condition in need of treatment. FIG. 5A shows sheath 20 soon after its retraction has begun, which exposes forward end 56 of scaffold 10. Forward end 56 is unconstrained by sheath 20, so it has begun to self-expand to a diameter greater than compressed diameter 16.

The terms “retract,” “retracted” and “retraction” refer to movement in rearward direction R. Retraction of sheath 20 may be performed by sliding entire sheath 20 rearward. Alternatively, retraction of sheath 20 may be performed by rolling back the forward end of sheath 20 such that the forward end slides rearward and on top of a stationary portion of sheath 20.

Guidewire 70 is not shown in FIGS. 5B-5L for ease of illustration, and it is to be understood that guidewire 70, if used, may be present during the segmental expansion process of FIGS. 5B-5L.

In FIG. 5B, sheath 20 has been retracted to expose front region 76 of scaffold 10. Sheath 20 has been retracted by distance 78 from its starting position in FIG. 2. Scaffold 10 is constrained from moving in the rearward direction by engagement of scaffold 10 with driver 36 and/or balloon 10. Driver 36 is configured to engage rear end 56 of scaffold 10. Driver 36 may include a disc that abuts rear end 56 and/or an expanding member, such as a compressed metal spring, that frictionally engages the interior surface of scaffold 10.

Driver 36 has been extended by distance 79 from its starting position in FIG. 2. In this context, the terms “extend,” “extended” and “extension” refer to movement in forward direction F. Extension of driver 36 may help reduce or prevent rearward movement of scaffold forward end 56 as front region 76 expands, so that scaffold 10 deploys within target region 74 (FIG. 5A). The axial lengths of front region 76 and balloon 26 may be similar to facilitate segmental expansion of the scaffold. For example, front region 76 (while at intermediate diameter 22 outside of sheath 20) may have an axial length that is from 90% to 110% of balloon axial length 52 (FIG. 2). Front region 76 is not constrained by sheath 20, so front region 76 has self-expanded to intermediate diameter 22. However, sheath 20 has caused stress relaxation to such an extent that front region 76 does not self-expand to nominal diameter 12 (FIG. 1A). Balloon 26 is disposed within front region 76 and will be used to radially expand front region 76.

In FIG. 5C, fluid has been forced into balloon 26, causing it to increase in diameter and apply radially outward pressure on the interior surface of front region 76. Balloon 26 may be inflated to an inflated diameter that is from 80% to 120% of nominal diameter 12 (FIG. 1A). For example, the inflated diameter may correspond to diameter 29 in FIG. 3B or 4B. The pressure applied by the balloon causes front region 76 to expand from intermediate diameter 22 to at least nominal diameter 12. Other regions of scaffold 10 remain within sheath 20 and do not radially expand to nominal diameter 12. The pressure from the balloon does not cause front region 76 to detach from the other regions of scaffold 10. Front region 76 is configured to remain attached to other regions of scaffold 10 during and after balloon inflation in FIG. 5C. Front region 76 has no filament that breaks off from another region of scaffold 10 as a result of balloon inflation in FIG. 5C.

Optionally, driver 36 may be extended during balloon inflation in FIG. 5C to help reduce or prevent rearward movement of scaffold forward end 56 as front region 76 is expanded by balloon 26, so that scaffold 10 deploys within target region 74 (FIG. 5A).

In FIG. 5D, balloon 26 is deflated. For example, balloon 26 may be deflated to diameter 31 of FIG. 3A, 3C, 4A or 4C. Front region 76 is configured to remain at 80% to 100% of nominal diameter 12 immediately after balloon 26 is deflated. Medial region 80 is adjacent to and disposed between front region 76 and rear region 82. Medial region 80 and rear region 82 remain at compressed diameter 16 since they are constrained within sheath 20.

In FIG. 5E, sheath 20 has been retracted to expose medial region 80 of scaffold 10. Sheath 20 has been retracted by distance 84 from its position in FIG. 5B. Scaffold 10 is constrained from moving in the rearward direction by engagement of scaffold 10 with driver 36. Driver 36 has been extended by distance 86 from its position in FIG. 5B. Extension of driver 36 may help reduce or prevent rearward movement of scaffold forward end 56 as medial region 80 expands, so that scaffold 10 deploys within target region 74 (FIG. 5A). The axial lengths of medial region 80 and balloon 26 may be similar to facilitate segmental expansion of the scaffold. For example, medial region 80 (while at intermediate diameter 22 outside of sheath 20) may have an axial length that is from 90% to 110% of balloon axial length 52 (FIG. 2). Medial region 80 is not constrained by sheath 20, so medial region 80 has self-expanded to intermediate diameter 22. However, sheath 20 has caused stress relaxation to such an extent that medial region 80 does not self-expand to nominal diameter 12. Balloon 26 will be used to radially expand medial region 80.

In FIG. 5F, balloon 26 is retracted from a position within front region 76 to a position within medial region 80. Balloon 26 is retracted while deflated.

In FIG. 5G, fluid has been forced into balloon 26, causing it to increase in diameter and apply radially outward pressure on the interior surface of medial region 80. Balloon 26 may be inflated as described for FIG. 5C. The pressure applied by the balloon causes medial region 80 to expand from intermediate diameter 22 to at least nominal diameter 12. Rear region 82 of scaffold 10 remains within sheath 20 and does not radially expand to nominal diameter 12. The pressure from the balloon does not cause medial region 80 to detach from rear region 82 or front region 76. Medial region 80 is configured to remain attached to other regions of scaffold 10 during and after balloon inflation in FIG. 5G. Medial region 80 has no filament that breaks off from another region of scaffold 10 as a result of balloon inflation in FIG. 5G.

Optionally, driver 36 may be extended during balloon inflation in FIG. 5G to help reduce or prevent rearward movement of scaffold forward end 56 as medial region 80 is expanded by balloon 26, so that scaffold 10 deploys within target region 74 (FIG. 5A).

In FIG. 5H, balloon 26 is deflated as described for FIG. 5D. Medial region 80 is configured to remain at 80% to 100% of nominal diameter 12 immediately after balloon 26 is deflated. Rear region 82 remains at compressed diameter 16 since it is constrained within sheath 20.

In FIG. 5I, sheath 20 has been retracted to expose rear region 82 of scaffold 10. Sheath 20 has been retracted by distance 88 from its position in FIG. 5E. Scaffold 10 is constrained from moving in the rearward direction by engagement of scaffold 10 with driver 36. Driver 36 has been extended by distance 90 from its position in FIG. 5E. Extension of driver 36 may help reduce or prevent rearward movement of scaffold forward end 56 as rear region 82 expands, so that scaffold 10 deploys within target region 74 (FIG. 5A).

The axial lengths of rear region 82 and balloon 26 may be similar to facilitate segmental expansion of the scaffold. For example, rear region 82 (while at intermediate diameter 22 outside of sheath 20) may have an axial length that is from 90% to 110% of balloon axial length 52 (FIG. 2).

Rear region 82 is not constrained by sheath 20, so rear region 82 has self-expanded to intermediate diameter 22. However, sheath 20 has caused stress relaxation to such an extent that rear region 82 does not self-expand to nominal diameter 12. Balloon 26 will be used to radially expand rear region 82.

In FIG. 5J, balloon 26 is retracted from a position within medial region 80 to a position within rear region 82.

In FIG. 5K, fluid has been forced into balloon 26, causing it to increase in diameter and apply radially outward pressure on the interior surface of rear region 82. Balloon 26 may be inflated as described for FIG. 5C. The pressure applied by the balloon causes rear region 82 to expand from intermediate diameter 22 to at least nominal diameter 12. The pressure from the balloon does not cause rear region 82 to detach from medial region 80. Rear region 82 is configured to remain attached to medial region 80 during and after balloon inflation in FIG. 5K. Rear region 82 has no filament that breaks off from another region of scaffold 10 as a result of balloon inflation in FIG. 5K.

Optionally, driver 36 may be extended during balloon inflation in FIG. 5K to help reduce or prevent rearward movement of scaffold forward end 56 as rear region 82 is expanded by balloon 26, so that scaffold 10 deploys within target region 74 (FIG. 5A).

In FIG. 5L, balloon 26 is deflated and scaffold 10 is shown completely deployed at target region 74. Rear region 76 is configured to remain at 80% to 100% of nominal diameter 12 immediately after balloon 26 is deflated. Front region 76, medial region 80, and rear region 76 remain connected to each other.

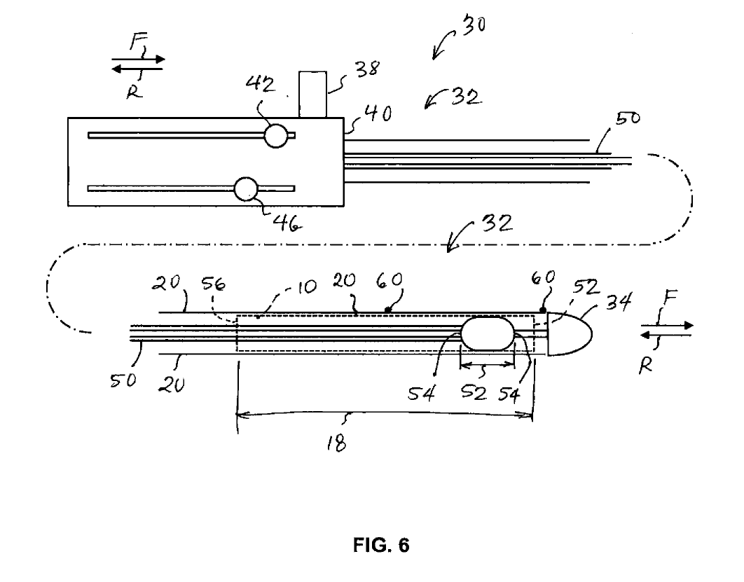

FIG. 6 shows example catheter system 30 similar to that of FIG. 4 except driver 36 and its knob 44 and driver connector 48 are absent. Instead of using driver 36, balloon 26 is used to help reduce or prevent rearward movement of scaffold forward end 56 as various regions of scaffold 10 are sequentially expanded, so that scaffold 10 deploys within the target region. In FIGS. 4 and 6, like reference numerals designate corresponding or like elements such that descriptions given for FIG. 4 also apply to FIG. 6 unless indicated otherwise.

Sheath knob 42 is operatively coupled to sheath 20. The user manipulates sheath knob 42 to control movements of sheath 20, as described above for FIG. 2. The forward and rearward movements of sheath 20 may be relative to balloon 26 and/or catheter tip 34. The forward and rearward movements of sheath 20 may be performed independently of movements of balloon 26.

Balloon knob 46 is operatively coupled to balloon 26. The user manipulates balloon knob 46 to control movements of balloon 26, as described above for FIG. 2. The forward and rearward movements of balloon 26 may be relative to sheath 20 and/or catheter tip 34. The forward and rearward movements of balloon 26 may be performed independently of movements of sheath 20.

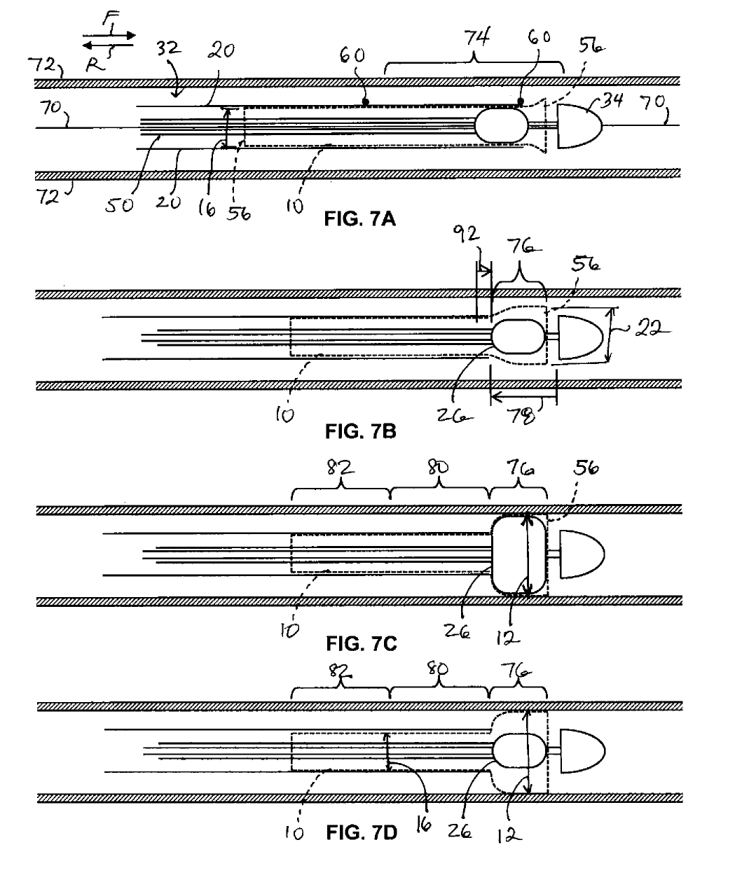

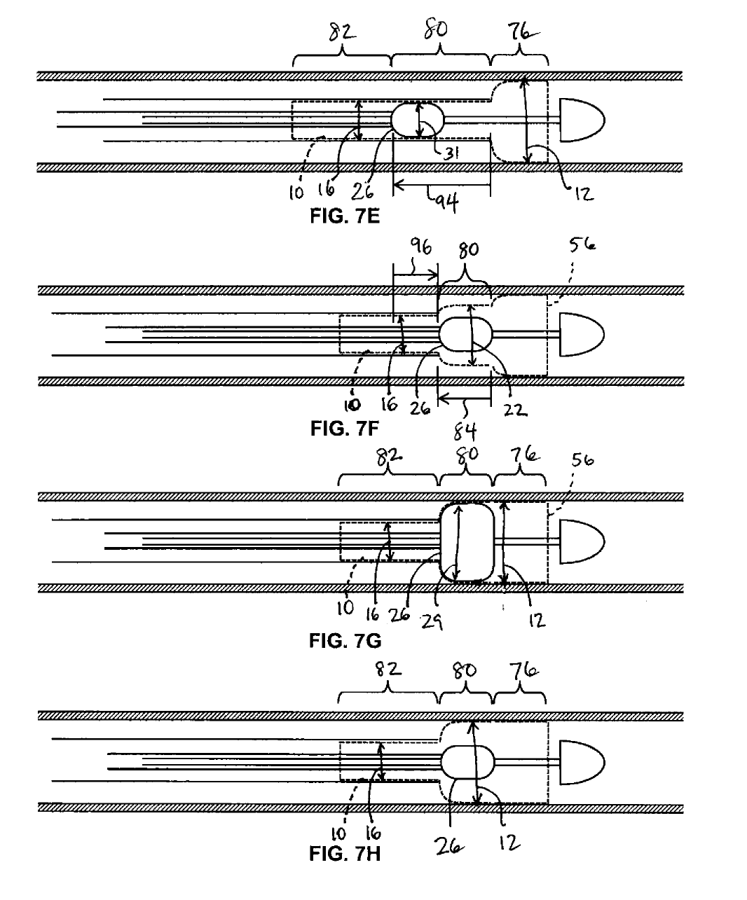

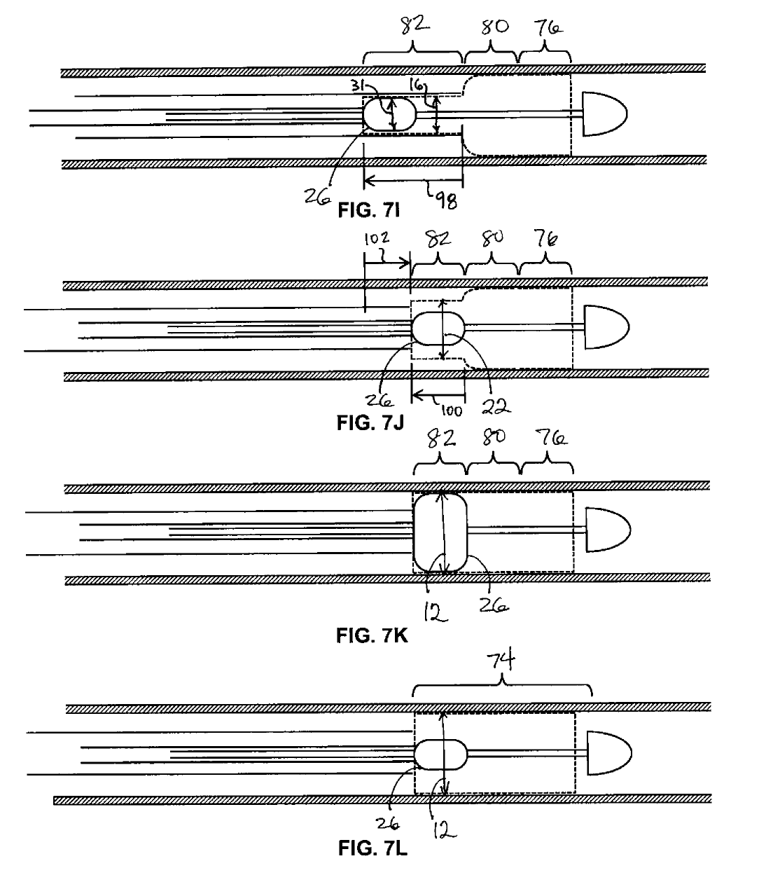

FIGS. 7A-7L illustrate an example process for segmental expansion of scaffold 10 using catheter assembly 32 of FIG. 6. In FIG. 7A, guidewire 70 was inserted in anatomical lumen 72 and has guided the forward end of catheter assembly 32 to target region 74. Although not illustrated, target region 74 may have a stenosis, lesion, or other condition in need of treatment. FIG. 7A shows sheath 20 soon after its retraction has begun, which exposes forward end 56 of scaffold 10. Forward end 56 has begun to self-expand to a diameter greater than compressed diameter 16.

Guidewire 70 is not shown in FIGS. 7B-7L for ease of illustration, and it is to be understood that guidewire 70, if used, may be present during the segmental expansion process of FIGS. 7B-7L.

In FIG. 7B, sheath 20 has been retracted to expose front region 76 of scaffold 10. Sheath 20 has been retracted away from front region 76. Sheath 20 has been retracted by distance 78 from its starting position in FIG. 6. Scaffold 10 is constrained from moving in the rearward direction by engagement of scaffold 10 with balloon 10.

In FIG. 7B, balloon 26 has been extended by distance 92 from its starting position in FIG. 6. Extension of balloon 26 may push scaffold 10 forward. Additionally or alternatively, extension of balloon 26 may help reduce or prevent rearward movement of scaffold forward end 56 while sheath 20 is retracted and while front region 76 expands, so that scaffold 10 deploys within target region 74 (FIG. 7A).

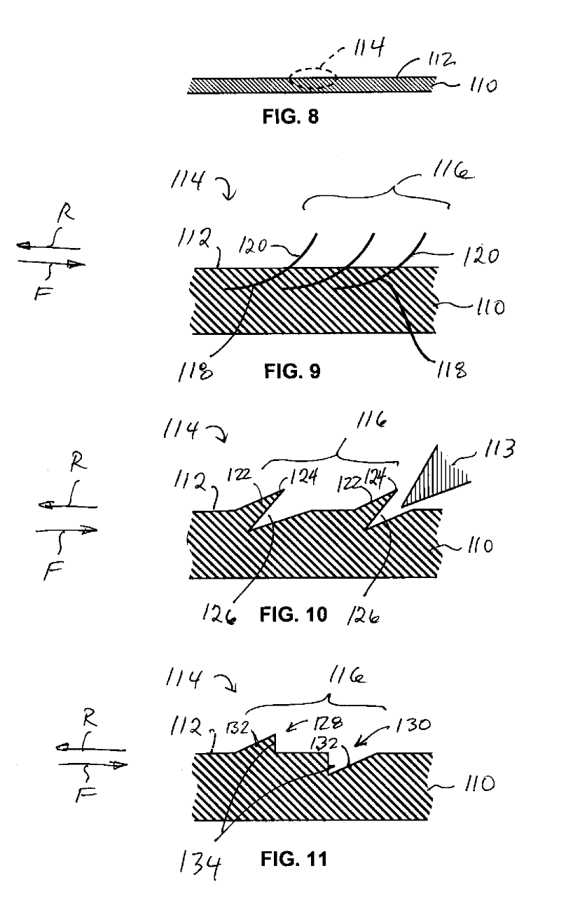

In some aspects, the outer surface of balloon 26 includes surface feature 116 (FIGS. 9-11) that engages scaffold 10. The surface feature produces a forward friction force (an example of a first forward friction force) on scaffold 10 when balloon 26 is extended by distance 92 and moves in a forward direction relative to sheath 20. The first forward friction force on front region 76 (an example of a first scaffold region) is any one or both of: (a) sufficient to push front region 76 out of sheath 20, and (b) sufficient to prevent front region 76 from moving rearward relative to balloon 26 while sheath 20 is retracted by distance 78.

The axial lengths of front region 76 and balloon 26 may be similar to facilitate segmental expansion of the scaffold. For example, the axial length of front region 76 may be as described for FIG. 5B. Front region 76 is not constrained by sheath 20, so front region 76 has self-expanded to intermediate diameter 22. However, sheath 20 has caused stress relaxation to such an extent that front region 76 does not self-expand to nominal diameter 12 (FIG. 1A). Balloon 26 is disposed within front region 76 and will be used to radially expand front region 76.

In FIG. 7C, fluid has been forced into balloon 26, causing it to increase in diameter and apply radially outward pressure on the interior surface of front region 76. Balloon 26 may be inflated as described for FIG. 5C. The effect on front region 76 and other regions of scaffold 10 are as described for FIG. 5C.

Optionally, balloon 26 may be extended during balloon inflation in FIG. 7C to help reduce or prevent rearward movement of scaffold forward end 56 as forward region 76 is expanded by balloon 26, so that scaffold 10 deploys within target region 74 (FIG. 7A).

In FIG. 7D, balloon 26 is deflated as described for FIG. 5D. The effect on front region 76 and other regions of scaffold 10 are as described for FIG. 5D. Balloon 26 is deflated to a deflated diameter that is less than or equal to scaffold compressed diameter 16. Doing so may control the amount of friction between balloon 26 and scaffold 10 during retraction of balloon into sheath 20, as will be discussed below.

As previously mentioned, balloon 26 may have pleats 100 to facilitate inflation and subsequent deflation. However, in some aspects, pleats 100 may not be able to fold sufficiently to allow balloon 26 to be retracted into sheath 20. The thickness of folds of material may allow balloon 26 to achieve the deflated configuration of FIG. 3C but not the configuration of FIG. 3A. In FIG. 3C, balloon 26 has deflated diameter 31 greater than compressed diameter 16. If deflated diameter 31 is more than 110% of scaffold compressed diameter 16, it may be too difficult for balloon 26 to be retracted into sheath 20. Also, if deflated diameter 31 is more than 110% of scaffold compressed diameter 16, an attempt to retract balloon 26 into sheath 20 may cause front region 76 to be pulled, at least partially, into sheath 20. To avoid these potential effects, balloon 26 may be configured as previously described for FIGS. 4A and 4B, where a balloon material having a high level of elasticity allows balloon 26 to expand to inflated diameter 29 and then deflate to deflated diameter 31 without folding. With folding absent, it may be possible for deflated diameter 31 to be less than or equal to scaffold compressed diameter 16, and balloon 26 may be retracted into sheath 20 without undue difficulty and without causing front region 76 to be pulled back into sheath 20.

In FIG. 7E, balloon 26 is retracted while scaffold 10 is in a partially covered state. Scaffold 10 is in a partially covered state since front region 76 is outside of sheath 20 while medial region 80 is inside of sheath 20. Balloon 26 is retracted by distance 94 from a position within front region 76 to a position within medial region 80. By doing so, balloon 26 is also retracted into sheath 20. Front region 76 remains outside of sheath 20 after balloon 26 is retracted into medial region 80.

Balloon 26 is retracted into medial region 80 while its deflated diameter 31 does not exceed 110% of scaffold compressed diameter 16. For example, deflated diameter 31 may be less than or equal to scaffold compressed diameter 16.

As previously discussed, the outer surface of balloon 26 may have surface feature 116 (FIGS. 9-11) that results in a forward friction force on front region 76 when balloon 26 is extended by distance 92 (FIG. 7B). Surface feature 116 may produce a rearward friction force on scaffold 10 when the balloon is retracted by distance 94. The rearward friction force on medial region 80 (an example of a second scaffold region) is insufficient to pull front region 76 (an example of a first scaffold region) into sheath 20.

In FIG. 7F, sheath 20 has been retracted to expose medial region 80 of scaffold 10. Sheath 20 has been retracted away from medial region 80. Sheath 20 has been retracted by distance 84 from its position in FIG. 7E. Scaffold 10 is constrained from moving in the rearward direction by engagement of scaffold 10 with balloon 26. Balloon 26 has been extended by distance 96 from its position in FIG. 7E. During at least a portion of extension distance 96, balloon 26 may engage scaffold 10 to push scaffold 10 forward. Extension of balloon 26 may help reduce or prevent rearward movement of scaffold forward end 56 as medial region 80 expands, so that scaffold 10 deploys within target region 74 (FIG. 7A).

The axial lengths of medial region 80 and balloon 26 may be similar to facilitate segmental expansion of the scaffold. For example, medial region 80 (while at intermediate diameter 22 outside of sheath 20) may have an axial length that is as described for FIG. 5E.

As previously discussed, the outer surface of balloon 26 may include surface feature 116 (FIGS. 9-11) that results in a rearward force on medial region 80 when balloon 26 is retracted by distance 94 in FIG. 7E. Surface feature 116 also produces a forward friction force (an example of a second forward friction force) on scaffold 10 when the balloon is moving forward out of sheath 20 while scaffold 10 is in a partially covered state, such as when balloon 26 is extended by distance 96 in FIG. 7F. The forward friction force (an example of a second forward friction force) is applied on medial region 80. The forward friction force is any one or both of: (a) sufficient to push medial region 80 out of sheath 20, and (b) sufficient to prevent medial region 80 from moving rearward relative to balloon 26 while sheath 20 is retracted by distance 84.

As a result of sheath retraction by distance 84 and/or balloon extension by distance 96, medial region 80 is not constrained by sheath 20, so medial region 80 has self-expanded to intermediate diameter 22. However, sheath 20 has caused stress relaxation to such an extent that medial region 80 does not self-expand to nominal diameter 12. Balloon 26 is disposed within medial region 80 and will be used to radially expand medial region 80.

In FIG. 7G, fluid has been forced into balloon 26, causing it to increase in diameter and apply radially outward pressure on the interior surface of medial region 80. Balloon 26 may be inflated as described for FIG. 7C. The effect on medial region 80 and other regions of scaffold 10 are as described for FIG. 5G.

Optionally, balloon 26 may be extended during balloon inflation in FIG. 7G to help reduce or prevent rearward movement of scaffold forward end 56 as medial region 80 is expanded by balloon 26, so that scaffold 10 deploys within target region 74 (FIG. 7A).

In FIG. 7H, balloon 26 is deflated as described for FIG. 7D. The effect on medial region 80 and other regions of scaffold 10 are as described for FIG. 5H.

In FIG. 7I, balloon 26 is retracted from a position within medial region 80 to a position within rear region 82. Balloon 26 is retracted while scaffold 10 is in a partially covered state. Scaffold 10 is in a partially covered state since medial region 80 is outside of sheath 20 while rear region 82 is inside of sheath 20. Balloon 26 is retracted by distance 98 into rear region 82. By doing so, balloon 26 is also retracted into sheath 20. Medial region 80 remains outside of sheath 20 after balloon 26 is retracted into rear region 82.

Balloon 26 is retracted into rear region 82 while its deflated diameter 31 does not exceed 110% of scaffold compressed diameter 16. For example, deflated diameter 31 may be less than or equal to scaffold compressed diameter 16.

The outer surface of balloon 26 may have surface feature 116 (FIGS. 9-11) having a rearward friction coefficient or mechanical engagement with scaffold 10 when the balloon is retracted by distance 94 (FIG. 7E). When the balloon is subsequently retracted by distance 98 (FIG. 7I), surface feature 116 may also have a rearward friction coefficient or mechanical engagement with scaffold 10 that produces a rearward force on rear region 82 (another example of a second scaffold region) that is insufficient to pull medial region 80 (another example of a first scaffold region) into sheath 20. These rearward friction coefficients (or associated rearward mechanical engagements) are less than the forward friction coefficients (or forward mechanical engagements) described herein.

In FIG. 7J, sheath 20 has been retracted to expose rear region 82 of scaffold 10. Sheath 20 has been retracted away from rear region 82. Sheath 20 has been retracted by distance 100 from its position in FIG. 7I. Scaffold 10 is constrained from moving in the rearward direction by engagement of scaffold 10 with balloon 26. Balloon 26 has been extended by distance 102 from its position in FIG. 7I. During at least a portion of extension distance 102, balloon 26 may engage scaffold 10 to push scaffold 10 forward. Extension of balloon 26 may help reduce or prevent rearward movement of scaffold forward end 56 as medial region 80 expands, so that scaffold 10 deploys within target region 74 (FIG. 7A).

The axial lengths of rear region 82 and balloon 26 may be similar to facilitate segmental expansion of the scaffold. For example, rear region 82 may have an axial length that is as described for FIG. 5I.

The outer surface of balloon 26 may include surface feature 116 (FIGS. 9-11) having a forward friction coefficient or mechanical engagement with scaffold 10 when the balloon is extended by distance 96 (FIG. 7F). When subsequently extending balloon 26 by distance 102 (FIG. 7J), surface feature 116 may have a forward friction coefficient or mechanical engagement that produces a forward friction force (another example of a second forward friction force) on rear region 82 (another example of a second scaffold region). The forward friction force is any one or both of: (a) sufficient to push rear region 82 out of sheath 20, and (b) sufficient to prevent rear region 82 from moving rearward relative to balloon 26 while sheath 20 is retracted by distance 100.

As a result of sheath retraction by distance 100 and/or balloon extension by distance 102, rear region 82 is not constrained by sheath 20, so rear region 82 has self-expanded to intermediate diameter 22. However, sheath 20 has caused stress relaxation to such an extent that rear region 82 does not self-expand to nominal diameter 12. Balloon 26 is disposed in rear region 82 and will be used to radially expand rear region 82.

In FIG. 7K, fluid has been forced into balloon 26, causing it to increase in diameter and apply radially outward pressure on the interior surface of rear region 82. Balloon 26 may be inflated as described for FIG. 7C. The effect on rear region 82 and other regions of scaffold 10 are as described for FIG. 5K.

Optionally, balloon 26 may be extended during balloon inflation in FIG. 7K to help reduce or prevent rearward movement of scaffold forward end 56 as forward region 76 is expanded by balloon 26, so that scaffold 10 deploys within target region 74 (FIG. 7A).

In FIG. 7L, balloon 26 is deflated and scaffold 10 is shown completely deployed at target region 74. Rear region 76 is configured to remain at 80% to 100% of nominal diameter 12 immediately after balloon 26 is deflated. Front region 76, medial region 80, and rear region 76 remain connected to each other.

Referring again to FIGS. 2 and 5A-5L, driver 36 is configured to engage the rear end of scaffold 10 to restrain rearward movement of scaffold 10 within sheath 20 and/or configured to engage the rear end of scaffold 10 to push scaffold 10 forward out of sheath 20.

In other aspects, FIGS. 2 and 5A-5L may be modified to include the use of balloon 26 to restrain rearward movement of scaffold 10 within sheath 20 and/or push scaffold 10 forward out of sheath 20. For example, both driver 36 and balloon 26 may be used to restrain rearward movement of scaffold 10 within sheath 20 and to push scaffold 10 forward out of sheath 20.

In FIGS. 6 and 7A-7L, there is no driver 36 configured to engage the rear end of scaffold 10 to restrain rearward movement of scaffold 10 within sheath 20, and no driver 36 configured to engage the rear end of scaffold 10 to push scaffold 10 forward out of sheath 20. Instead of driver 36, balloon 26 is used to restrain rearward movement of scaffold 10 within sheath 20 and/or push scaffold 10 forward out of sheath 20.

In other aspects, FIGS. 6 and 7A-7L may be modified to include a driver configured to engage the rear end of scaffold 10 to restrain rearward movement of scaffold 10 within sheath 20 and/or configured to engage the rear end of scaffold 10 to push scaffold 10 forward out of sheath 20. That is, FIGS. 6 and 7A-7L may be modified to include driver 36 and its knob 44 and driver connector 48. For example, both driver 36 and balloon 26 may be used to restrain rearward movement of scaffold 10 within sheath 20 and to push scaffold 10 forward out of sheath 20.

As shown in FIG. 8, balloon 26 includes balloon wall 110 made of elastic polymer material. Balloon wall 110 is configured to stretch elastically to increase in surface area by at least 200% when balloon 26 is inflated. For example, C-Flex (R) thermoplastic elastomer, silicone rubber, and other materials may be used to allow for the minimum 200% expansion. Balloon wall 110 forms outer surface 112 of balloon 26. Diameters 27, 29, and 31 (FIGS. 3A to 4C) are measured from outer surface 112. Region 114 of balloon wall 110 includes surface feature 116 (FIGS. 9-11) that provide the forward and rearward friction coefficients described above. Surface feature 116 may be located at multiple regions of balloon 26.

In FIG. 9, balloon 26 includes flexible fibers 118 partially embedded in balloon wall 110. Each fiber 118 has exposed portion 120 outside of balloon wall 110 and protruding radially outward from balloon outer surface 112. Exposed portions 120 collectively form surface feature 116. Each exposed portion 120 points in forward direction F. When balloon 26 is retracted into scaffold 10 in rearward direction R (for example, distances 94 and 98 in FIGS. 7E and 7I), scaffold 10 pushes exposed portions 120 down toward balloon outer surface 112. Exposed portions 120 bend toward balloon outer surface 112, which results in the rearward friction coefficients described above. When balloon 26 is extended in forward direction F (for example, distances 92, 96, and 102 in FIGS. 7B, 7E, and 7J), scaffold 10 lifts exposed portions 120 away from balloon outer surface 112, which results in the forward friction coefficients described above. Exposed portions 120 do not bend toward balloon outer surface 112 when balloon 26 is extended. The difference in bending response by exposed portions 120 causes the rearward friction force to be less than the forward friction force.

In FIG. 10, balloon wall 114 includes skived portions 122 having ends 124 oriented toward a forward end of the balloon. Skived portions 122 collectively form surface feature 116. Each skived portion 122 is a portion of balloon outer surface 112 that has been lifted and bent up from surrounding portions of balloon outer surface 112. Skived portions 122 may be formed by a skiving machine having blade 113 that enters balloon outer surface 112 at an acute angle while moving in rearward direction R. Blade 113 causes a portion of balloon outer surface 112 to lift up and form depression 126, thereby forming skived portion 122 having depression 126 located at its base.

When balloon 26 is retracted into scaffold 10 in rearward direction R (for example, distances 94 and 98 in FIGS. 7E and 7I), scaffold 10 pushes skived portions 122 down toward balloon outer surface 112. Depressions 126 allow skived portions 122 to bend toward balloon outer surface 112, which results in the rearward friction coefficients described above. When balloon 26 is extended in forward direction F (for example, distances 92, 96, and 102 in FIGS. 7B, 7E, and 7J), scaffold 10 lifts skived portions 122 away from balloon outer surface 112, which results in the mechanical engagement described above. Skived portions 122 do not bend toward balloon outer surface 112 when balloon 26 is extended. The difference in bending response by skived portions 122 causes the rearward friction coefficients to be less than the mechanical engagement.

In FIG. 11, surface feature 116 comprises any one or a combination of rib 128 protruding from balloon outer surface 112 and indentation 130 into balloon outer surface 112. Rib 128 and indentation 130 include ramp surfaces 132 that face in rearward direction R and catch surfaces 134 that face in forward direction F. When balloon 26 is retracted into scaffold 10 in rearward direction R (for example, distances 94 and 98 in FIGS. 7E and 7I), ramp surfaces 132 function like wedges that causes scaffold 10 to deflect and/or cause balloon wall 110 to deflect, thereby allowing the balloon to retract with the rearward friction coefficients described above. The deflection allows scaffold 10 to slide over ramp surfaces 132. When balloon 26 is extended in forward direction F (for example, distances 92, 96, and 102 in FIGS. 7B, 7E, and 7J), catch surfaces 134 engage scaffold 10, which result in the mechanical engagement described above. The difference in response (sliding versus engagement) between the ramp and catch surfaces causes the rearward friction coefficients to be less than the forward friction coefficients.

Balloon outer surface 112 may include multiple ribs and/or multiple indentations. Each rib 132 and/or indentation 132 may be formed by blow molding of balloon wall 110 in a mold cavity having correspondingly shaped recesses to form rib 132 and/or correspondingly shaped protrusions to form indentation 132.

Other types of surface features may be implemented on balloon outer surface 112 to produce friction during balloon retraction that is greater than friction during balloon extension. For example, a spongy material may be applied to balloon outer surface 112 to produce the difference in friction. Also, the various types of surface features described herein may be combined on a single balloon.

In other aspects, the catheter and method described above may be modified such that sheath 20 is not retracted to expose scaffold 10, or driver 36 is not extended to expose scaffold 10. Retracting sheath 20 or extending driver 36 forward may cause scaffold 10 to become axially compressed, which results in a tendency of scaffold 10 to increase in diameter, which in turn may increase friction between scaffold 10 and sheath 20. This increase in friction may be avoided by pulling scaffold 10 to expose scaffold 10 instead of relying primarily on retracting sheath 20 and/or extending driver 36 forward to expose scaffold 10.

To expose scaffold 10, surface feature 116 of balloon 26 is used to frictionally engage scaffold 10 and to pull forward region 76 out of sheath 20. Next, balloon 26 is inflated to expand forward region 76 to nominal diameter 12 while medial region 80 remains at compressed diameter 16 inside of sheath 20. Optionally, balloon 26 is deflated and retracted into sheath 20 and medial region 80, surface feature 116 is used to frictionally engage scaffold 10 and to pull medial region 80 out of sheath 20. Next, balloon 26 is inflated to expand medial region 80 to nominal diameter 12 while rear region 82 remains at compressed diameter 16 inside of sheath 20. Optionally, balloon 26 is deflated and retracted into sheath 20 and rear region 82, surface feature 116 is used to frictionally engage scaffold 10 and to pull rear region 82 out of sheath 20. Next, balloon 26 is inflated to expand rear region 82 to nominal diameter 12.

Additionally or alternatively, scaffold 10 is exposed by using pulling device 140 adjacent to balloon 26. Pulling device 140 may be secured to inflation tube 142 that conveys inflation fluid from fluid port 38 to balloon 26. As shown in FIG. 12, pulling device 140 can be located distal or in front of balloon 26. As shown in FIG. 13, pulling device 140 can be located proximal or to the rear of balloon 26. To enable pulling of scaffold 10, pulling device 140 may include a surface feature that is the same as surface feature 116 described above for balloon 26. Optionally, with pulling device 140 present, balloon 26 does not have surface feature 116.

To expose scaffold 10, the surface feature of pulling device 140 is used to frictionally engage scaffold 10 and to pull forward region 76 out of sheath 20. Next, balloon 26 is inflated to expand forward region 76 to nominal diameter 12 while medial region 80 remains at compressed diameter 16 inside of sheath 20. Optionally, balloon 26 is deflated and retracted into sheath 20 and medial region 80, the surface feature of pulling device 140 is used to frictionally engage scaffold 10 and to pull medial region 80 out of sheath 20. Next, balloon 26 is inflated to expand medial region 80 to nominal diameter 12 while rear region 82 remains at compressed diameter 16 inside of sheath 20. Optionally, balloon 26 is deflated and retracted into sheath 20 and rear region 82, the surface feature of pulling device 140 is used to frictionally engage scaffold 10 and to pull rear region 82 out of sheath 20. Next, balloon 26 is inflated to expand rear region 82 to nominal diameter 12.

As discussed above, scaffold 10 may be formed of a bioresorbable polymer material. Suitable bioresorbable polymer materials include, without limitation, poly(L-lactide) (“PLLA”), poly(glycolide) (PGA), polycaprolactone (PCL), poly(trimethylene carbonate) (PTMC), polydioxanone (PDO), poly(4-hydroxy butyrate) (P4HB), poly(butylene succinate) (PBS), poly(D-lactide), poly(DL-lactide),poly(L-lactide-co-glycolide) (“PLGA”), poly(D-lactide-co-glycolide) or poly(L-lactide-co-D-lactide) (“PLLA-co-PDLA”) with less than 10% D-lactide, PLLD/PDLA stereo complex, aliphatic polyester, and any combination in any proportion thereof. Scaffold 10 may be formed of a combination of bioresorbable polymer material and metal. For example, some filaments 11 of scaffold 10 may be formed bioresorbable polymer material and other filaments 11 of scaffold 10 may be formed of metal. Suitable metals include, without limitation, nickel titanium (NiTi), nickel-chromium, and other alloys.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A catheter system comprising:
a hollow tube forming a sheath;
a balloon configured to slide out of and into the sheath;
a braid of polymer filaments forming a scaffold, the scaffold including a first scaffold region and a second scaffold region located rearward of the first scaffold region, the scaffold having a covered state when fully inside the sheath, a partially covered state when the first scaffold region is outside the sheath and the second scaffold region is inside the sheath, and a non-covered state when fully outside the sheath,
wherein when in the covered state, the first scaffold region and the second scaffold region are disposed within the sheath and each have starting diameters, and
wherein when in the partially covered state, the first scaffold region is disposed outside of the sheath and connected to the second scaffold region, the second scaffold region is disposed within the sheath, and the balloon is configured to inflate while outside of the sheath such that inflation expands the first scaffold region to a diameter greater than the starting diameter of the first scaffold region.

2. The catheter system of claim 1, wherein when in the non-covered state, the first scaffold region and the second scaffold region remain connected to each other and are disposed outside of the sheath, and the balloon is configured to inflate while outside of the sheath such that inflation expands the second scaffold region to a diameter greater than the starting diameter of the second scaffold region.

3. The catheter system of claim 1, wherein:
the balloon includes a balloon forward end and a balloon rear end, the balloon has a balloon axial length from the balloon rear end to the balloon forward end,
the scaffold includes a scaffold forward end and a scaffold rear end, the scaffold has a scaffold axial length from the scaffold rear end to the scaffold forward end, and
the scaffold axial length is greater than the balloon axial length.

4. The catheter system of claim 1, wherein, after the balloon was already inflated to expand the first scaffold region, the balloon is configured to deflate to a deflated diameter while the scaffold is in the partially covered state, and the deflated diameter allows the balloon to be pulled into the sheath and into the second scaffold region.

5. The catheter system of claim 1, wherein the balloon includes an outer surface and a surface feature on the outer surface, the surface feature produces a rearward friction force on the second scaffold region when the balloon is pulled in a rearward direction relative to the second scaffold region while the scaffold is in the partially covered state, and the rearward friction force on the second scaffold region is insufficient to pull the first scaffold region into the sheath when the scaffold is in the partially covered state.

6. The catheter system of claim 5, wherein the surface feature produces a forward friction force on the first scaffold region when moving in a forward direction relative to the sheath while the scaffold is in the covered state, and the first forward friction force on the first scaffold region is any one or both of: (a) sufficient to push the first scaffold region out of the sheath, and (b) sufficient to prevent the first scaffold region from moving rearward relative to the balloon while the sheath is retracted away from the first scaffold region.

7. The catheter system of claim 5, wherein the surface feature produces a second forward friction force on the second scaffold region when the balloon is pushed in a forward direction relative to the sheath while the scaffold is in the partially covered state, and the second forward friction force on the second scaffold region is any one or both of: (a) sufficient to push the second scaffold region out of the sheath, and (b) sufficient to prevent the second scaffold region from moving rearward relative to the balloon while the sheath is retracted away from the first scaffold region.

8. The catheter system of claim 5, wherein the balloon includes a balloon wall and fibers in the balloon wall, each fiber having an a portion exposed outside of the balloon wall, and the exposed portions collectively form the surface feature.

9. The catheter system of claim 5, wherein the balloon includes a balloon wall, the balloon wall having skived portions having ends oriented toward a forward end of the balloon, and the skived portions collectively form the surface feature.

10. The catheter system of claim 5, wherein the surface feature is formed by any one or a combination of a rib protruding from the outer surface of the balloon and an indentation into the outer surface of the balloon.

11. The catheter system of claim 1, wherein the sheath is configured to retract in a rearward direction relative to the scaffold while the scaffold is in the covered state, and at least a portion of the retraction places the scaffold in the partially covered state to allow the inflation of the balloon that expands the first scaffold region to the diameter greater than the starting diameter of the first scaffold region.

12. The catheter system of claim 11, wherein the sheath is configured to further retract in the rearward direction relative to scaffold while the scaffold is in the partially covered state and after inflation of the balloon has expanded the first scaffold region, and at least a portion of the further retraction places the scaffold in the non-covered state to allow inflation of the balloon that expands the second scaffold region to the diameter greater than the starting diameter of the second scaffold region.

13. The catheter system of claim 1, further comprising driver disposed within the sheath, the driver configure to engage the scaffold while the scaffold is in the partially covered state, the engagement sufficient to prevent the first scaffold region from being pulled into the sheath when the sheath is retracted away from the first scaffold region or when the balloon is pulled into the second scaffold region.

14. A method of implanting a scaffold, the method comprising:
inserting the catheter system of claim 1 into an anatomical lumen; followed by
retracting the sheath to expose the first region of the scaffold; followed by
inflating the balloon to expand the first region while the second region of the scaffold is disposed within the sheath; followed by
retracting the sheath to expose the second region; followed by
inflating the balloon to expand the second region; followed by
withdrawing the catheter assembly from the anatomical lumen while the first and second regions remain attached to each other and remain in the anatomical lumen.

15. A method of deploying a scaffold, the method comprising:
exposing a first region of a scaffold out from within the sheath; followed by
inflating a balloon to expand the first region while a second region of the scaffold is disposed in the sheath; followed by
exposing the second region of the scaffold out from within the sheath; followed by
inflating the balloon to expand the second region while the second region remains connected to the first region.

16. The method of claim 15, wherein when exposing the first region of the scaffold, the balloon engages the first region to prevent movement of the first region in a rearward direction into the sheath.

17. The method of claim 15, wherein after inflating the balloon to expand the first region and before inflating the balloon to expand the second region, the method further comprises deflating the balloon and then retracting the balloon into the second region while the second region is disposed within the sheath.

18. The method of claim 17, wherein after retracting the balloon into the second region and before inflating the balloon to expand the second region, the method further comprises pushing the second region out of the sheath by extending the balloon in a forward direction out of the sheath while the balloon engages the second region.

19. The method of claim 15, wherein when exposing the second region of the scaffold, the balloon engages the second region to prevent movement of the second region in a rearward direction into the sheath.

20. The method of claim 15, wherein after inflating the balloon to expand the second region, the method further comprises:
exposing a rear region of the scaffold out from the sheath; followed by
inflating the balloon to expand the rear region while the rear region remains connected to the second region.

21-23. (canceled)
